(19) United States
(12) Patent Application Publication
(10) Pub. No.: US 2023/0120384 A1
(43) Pub. Date: Apr. 20, 2023

(54) MENTHOL- AND MAGNESIUM-INFUSED KINESIOLOGY TAPE

(71) Applicants: Heather Sloan, Sarasota, FL (US); Enwei Li, Richmond Hill (CA)

(72) Inventors: Heather Sloan, Sarasota, FL (US); Enwei Li, Richmond Hill (CA)

(21) Appl. No.: 17/938,364

(22) Filed: Oct. 6, 2022

Related U.S. Application Data
(60) Provisional application No. 63/262,699, filed on Oct. 19, 2021.

(51) Int. Cl. A61L 15/44; A61F 13/02

(52) U.S. Cl. CPC A61L 15/44; A61F 13/0283; A61L 2300/102; A61L 2300/216; A61L 2300/402; A61L 2300/45

(57) ABSTRACT

Magnesium-infused and menthol-infused kinesiology tape is disclosed. The tape comprises an absorbent fabric substrate with adhesive on one side of the substrate to be applied to a person's skin and includes magnesium salt and menthol for promoting pain relief while the tape adheres to the person's skin.

### CROSS-REFERENCE TO RELATED APPLICATION

This nonprovisional patent application is based on and claims the benefit of priority of U.S. provisional application No. 63/262,699, filed Oct. 19, 2021, the contents including figures of which are hereby incorporated by reference in its entirety.

### FIELD

The present subject matter relates, in general, to kinesiology tape and, more particularly, to menthol-impregnated/infused and magnesium-impregnated/infused kinesiology tape.

### BACKGROUND OF THE INVENTION

Kinesiology tape, a cloth-based self-adhesive tape, has anisotropic stretch properties, enabling such tapes to stretch principally in one direction, such as length, rather than the other direction, such as width. Traditionally, kinesiology tapes have been used to treat muscles and/or connective tissues that were stressed, due to physical activity, where there is no open wound. Kinesiology tape is applied to an individual in a stretched condition to provide a therapeutic benefit to the individual from the “recoil” (i.e., elastic) property of various tapes. Kinesiology tapes for treating athletic injuries and physical disorders are known. See, e.g., U.S. Pat. No. 10,973,697 to Quinn; U.S. Pat. No. 10,869,776 to Hahn et al.; U.S. Pat. No. 10,517,767 to Capobianco et al.; U.S. Pat. No. 10,342,889 to Fladoos; and U.S. Pat. No. 8,216,415 to Quinn. Kinesiology tapes are thin, stretchy, elastic cotton strips provided with acrylic adhesives. Conventional therapeutic kinesiology tape is used to treat inflammation as well as a variety of musculoskeletal and sports injuries.

To relieve pain, Marx in U.S. Pat. No. 5,898,037 teaches adding ionic forms of magnesium to liquids while Mont in U.S. Pat. No. 4,059,690 teaches adding menthol to liquids and ointments. U.S. Pat. No. 10,751,299 to Ghalili discloses hydrogel compositions including menthol for use as topical oral analgesics. Massaging magnesium oil into skin is known to increase transport of the magnesium across the skin. See FIGS. 1A and 1B, reproduced from Heard, C. (2011). “In vitro transdermal delivery of magnesium.” 1-2. Menthol is known to enhance magnesium ion transport across skin; see FIGS. 2A and 2B reproduced from Turunen, et al. (2019). “The permeability of magnesium across the skin is enhanced by menthol cream.” Magnesium Research, 32 (4), 118-123. However, kinesiology tapes used for musculoskeletal stability cannot be combined with pain creams, as the wet nature of a pain cream will interfere with the adhesive properties of a tape, resulting in a tape not adhering to one's skin.

Impregnation of a substrate such as cotton with a metal salt is known in other arts, as disclosed in U.S. Pat. No. 6,905,520 to Auger. However, Auger does not combine the metal salt with menthol and does not at all suggest adhering the impregnated substrate to a subject.

The contents of the literature cited herein, including patents, is incorporated by reference in its entirety.

As can be appreciated, there is a need for a kinesiology tape that includes a combination of a magnesium composition and menthol, effective pain-relieving ingredients that in combination improve transport through the skin.

### SUMMARY OF THE INVENTION

One aspect of the present subject matter is directed to a kinesiology tape comprising a substrate; an amount of an adhesive, adhered to a surface of the substrate that is to be applied to a person's skin, effective to adhere the substrate to the person's skin; and effective relative amounts of magnesium salt and menthol, on the substrate (12), operative to promote pain relief.

Another aspect of the present subject matter is directed to a method of manufacturing a kinesiology tape, comprising providing an absorbent fabric substrate; blending a magnesium component and a menthol component into an adhesive to form an infused adhesive; and applying the infused adhesive to the absorbent fabric substrate.

The present subject matter thus provides pain-relieving ingredients including menthol and magnesium salts, combined with an adhesive infused into a kinesiology tape. The tape fabric provides a stable substrate for the pain-relieving ingredients, which are operative to promote pain relief and healing while adhering to skin, providing a unique healing process.

These and other features, aspects, and advantages of the present subject matter will become better understood with reference to the following description, drawings, and claims.

### BRIEF DESCRIPTION OF THE DRAWINGS

FIG. 1A is a prior art chart illustrating transport of magnesium across skin over time;

FIG. 1B is a prior art chart illustrating transport of magnesium in the presence of aloe and menthol across skin over time;

FIG. 2A is a prior art chart illustrating transport of magnesium ions in the presence of levomenthol across skin over time;

FIG. 2B is a prior art chart illustrating transport of magnesium ions alone across skin over time;

FIG. 3 is a perspective view of a roll of kinesiology tape illustrating an embodiment of the present subject matter; and

FIG. 4 depicts an adhesive surface of a portion thereof, on an enlarged scale relative to FIG. 3.

Throughout the drawing figures and detailed description, similar reference numerals shall be used to refer to similar components of the present subject matter.

### DETAILED DESCRIPTION OF THE INVENTION

The following description is the best currently contemplated mode of carrying out exemplary embodiments of the invention. The description is not to be taken in a limiting sense but is made merely for purposes of illustrating general principles of the invention, since the scope of the invention is best defined by the appended claims.

An embodiment of the present subject matter is a magnesium-infused or impregnated and menthol-infused or impregnated kinesiology tape. The infused magnesium- and menthol-ingredients are synergistically absorbed transdermally through a person's skin into the person's body while the kinesiology tape provides stability.

Tape-infused menthol is absorbed into skin receptors, and pain signals to the brain are temporarily blocked. Menthol provides temporary pain relief by providing a cooling sensation that blocks nerve endings from sending pain signals to the brain. The kinesiology tape comprises an amount of within a safe range of menthol as determined by FDA and Health Canada. As used herein, an effective amount is indicative of an amount that improves the permeation of magnesium across skin and provides some relief of discomfort when applied to the skin. Menthol is generally blended into the adhesive in an amount of about 1.25 weight % to about 16 weight % of the adhesive blend. However, in some cases, menthol may be present in amounts up to about 50 weight % of the adhesive blend.

The magnesium infused into the tape not only helps to relieve muscle spasms but also helps to provide a faster healing process for the muscles. In addition, the magnesium ions help to flush lactic acid from an injured area or overused muscle.

The kinesiology tape comprises an amount of a magnesium salt within a safe range of magnesium chloride as determined by the United States Food and Drug Administration (FDA) and Health Canada. As used herein, an amount is indicative of an amount that provides some relief of discomfort when applied to the skin. Magnesium is generally blended into the adhesive in an amount of about 1.25 weight % to about 16 weight % of the adhesive blend. However, in some cases, the magnesium may be present in amounts up to about 50 weight % of the adhesive blend.

Kinesiology tape of the present subject matter is not limited to the magnesium salt and menthol ingredients. Other active ingredients may be infused therein (along with the magnesium and menthol combination) as well.

The present invention is not limited to kinesiology tape. In some embodiments, a blend of adhesive, menthol, magnesium, and optionally additional ingredients may be applied to a fabric substrate, where the fabric substrate is not particularly limited.

The shape and size of the impregnated or infused kinesiology tape is not particularly limited. In some embodiments, the kinesiology tape has a rolled configuration, which may be unrolled for application. In other embodiments, the kinesiology tape may be cut in predetermined shapes and sizes.

The kinesiology tape may be provided in a variety of preselected patterns and preselected colors.

The materials of manufacture are not particularly limited. The substrate may be any suitable fabric known in the art, such as cotton, silk, or a synthetic material. Suitable adhesives or glues will not lose their adhesive properties when in contact with menthol and magnesium salts including magnesium chloride.

In some embodiments, the adhesive surface of the tape may have a backing paper or release layer.

In addition to manufacturing processes incorporated by reference, kinesiology tape of the present subject matter may be manufactured by combining menthol, a suitable magnesium salt, e.g., magnesium chloride, and a suitable adhesive, and applying the combined composition onto a fabric substrate. Generally, the adhesive and the accompanying ingredients infuse into the substrate prior to drying or solidifying.

Magnesium-infused or impregnated and menthol-infused or impregnated kinesiology tape of the present subject matter may be used as follows. A person cleans and prepares a predetermined area of skin so that it is, e.g., oil free and lotion free. Next, paper backing is removed to expose an adhesive layer on the tape. Then, the adhesive-layered surface of the tape is applied, with a slight stretch of the tape, to the person's skin. Lastly, skin-applied tape is rubbed (small amount of pressure to the surface) to increase adhesion. Tape may be worn for up to 1 week including during showering.

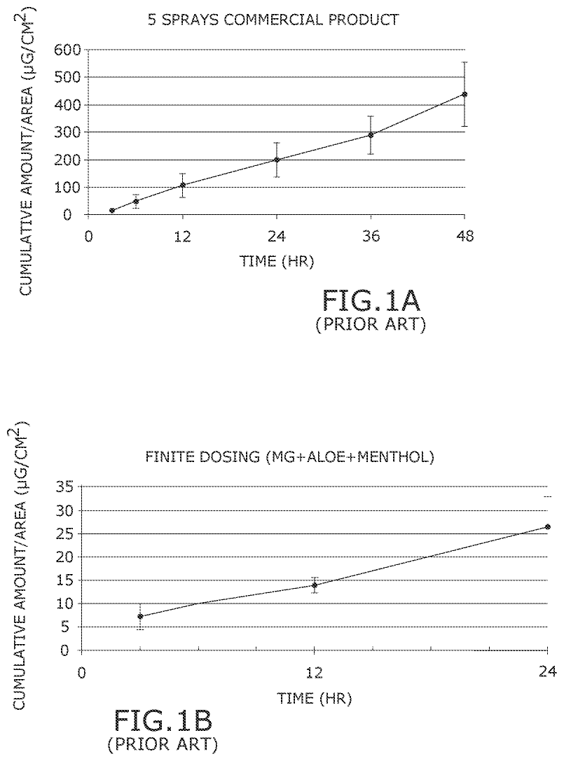

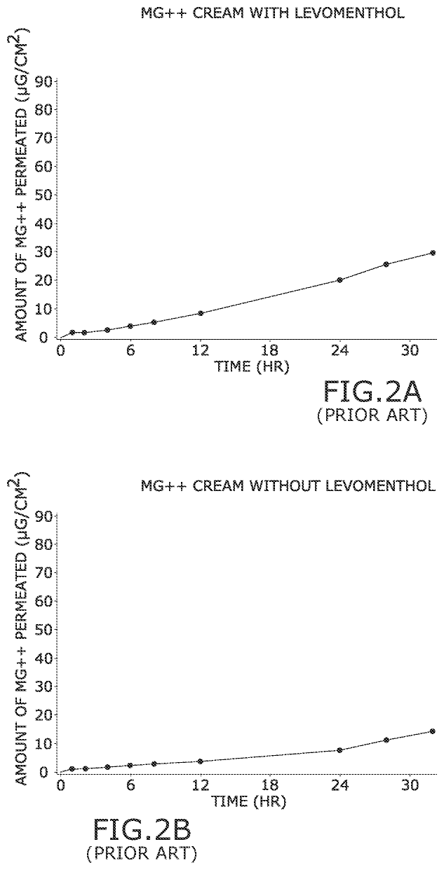

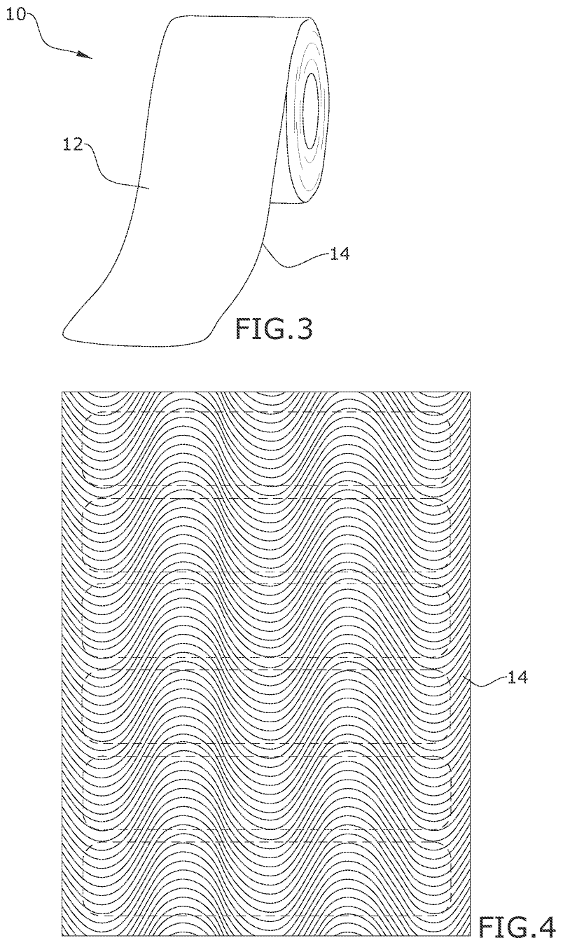

Referring to FIGS. 1A, 1B, 2A, 2B, 3, and 4, FIGS. 1A-1B and 2A-2B illustrate permeation of magnesium across skin according to published literature discussed in the background. FIGS. 3 and 4 illustrate a roll of magnesium-infused or impregnated and menthol-infused or impregnated kinesiology tape 10 according to an embodiment of the present invention. The tape 10 comprises a suitably absorbent fabric substrate 12 with an adhesive 14 applied to a surface of the absorbent fabric substrate 12 selected to be applied to one's skin. The adhesive 14 and/or the fabric substrate 12 comprise relative amounts of magnesium salt and menthol effective to promote pain relief while the tape adheres to the skin. Note that the adhesive 14 as shown is applied in a repeating wave pattern.

Illustrated and described in this patent application is a menthol-infused or impregnated and magnesium-infused or impregnated kinesiology tape. While the present subject matter has been described in reference to a single embodiment, the present subject matter is not limited to the current embodiment. On the contrary, many alternatives, changes, and/or modifications will become apparent to those of ordinary skill in the field of the present subject matter after this application has been read. Therefore, all such alternatives, changes, and modifications are to be viewed as part of the present subject matter insofar as they fall within the spirit and scope of the following claims.

What is claimed is:

1. A kinesiology tape (10) comprising:
a substrate (12);
an amount of an adhesive (14), adhered to a surface of the substrate (12) that is to be applied to a person's skin, effective to adhere the substrate to the person's skin; and
effective relative amounts of magnesium salt and menthol, on the substrate (12), operative to promote pain relief.

2. The kinesiology tape (10) of claim 1, wherein the adhesive is a blend comprising the menthol in an amount of about 1.25 weight % to about 50 weight % of the blend.

3. The kinesiology tape (10) of claim 1, wherein the adhesive is a blend comprising the magnesium salt in an amount of about 1.25 weight % to about 50 weight % of the blend.

4. The kinesiology tape (10) of claim 1, wherein the kinesiology tape (10) is made of a preselected color and is also made to include a preselected pattern.

5. The kinesiology tape (10) of claim 1, wherein the substrate (12) comprises an absorbent fabric.

6. The kinesiology tape (10) of claim 5, wherein the absorbent fabric is selected from the group consisting of: cotton, silk, a synthetic material, and any combination thereof.

7. The kinesiology tape (10) of claim 5, wherein the magnesium salt and the menthol are present within the adhesive and the adhesive is infused into or onto the absorbent fabric.

8. The kinesiology tape (10) of claim 5, wherein the magnesium salt and the menthol are infused into or onto the absorbent fabric.

9. The kinesiology tape (10) of claim 5, wherein the kinesiology tape (10) is configured as a roll.

10. The kinesiology tape (10) of claim 5, wherein the kinesiology tape (10) has a predetermined shape.

11. A method of manufacturing a menthol-infused and magnesium-infused adhesive fabric article, comprising:
providing an absorbent fabric substrate;
blending a magnesium component and a menthol component into an adhesive to form an adhesive blend; and
applying the adhesive blend to the absorbent fabric substrate.

12. The method of claim 11, wherein the blending step blends additional ingredients into the adhesive to form the adhesive blend.

13. The method of claim 11, further comprising, prior to blending, providing the magnesium component in an amount of about 1.25 weight % to about 50 weight % of the adhesive blend and the menthol component in an amount of about 1.25 weight % to about 50 weight % of the adhesive blend.

14. The method of claim 11, wherein the adhesive fabric article is a kinesiology tape, further comprising, after applying the adhesive blend, cutting the absorbent fabric substrate to a predetermined shape.

15. The method of claim 14, wherein the predetermined shape is a strip.

16. The method of claim 15, further comprising rolling the strip into a roll.
